(19) United States
(12) Patent Application Publication
(10) Pub. No.: US 2026/0354862 A1
(43) Pub. Date: Nov. 12, 2026

(54) EXPANDABLE MEDICAL DEVICE

(71) Applicant: XTRAC O.S LTD, Raanana (IL)

(72) Inventor: Uri STEIN, Ramat Gan (IL)

(21) Appl. No.: 19/470,433

(22) PCT Filed: Mar. 25, 2024
(86) PCT No.: PCT/IL2024/050302

§ 371 Date: Sep. 29, 2025

(30) Foreign Application Priority Data
Sep. 29, 2023 (IL) 301797

(51) Int. Cl. A61B 17/34; A61B 17/00; A61B 17/02; A61N 1/05; A61N 1/362; A61N 1/375

(52) U.S. Cl. CPC A61B 17/3439; A61B 17/3468; A61N 1/056; A61N 1/362; A61N 1/37516; A61B 2017/00477; A61B 2017/0225

(57) ABSTRACT

The present disclosure provides a medical device that is capable of performing a controlled expansion of a portion thereof during a medical procedure. The medical device is intended to be inserted into a lumen of interest during the medical procedure, for example a lumen of a blood vessel or a lumen of a lead of a cardiac implemented electronic device (CIED). Once the medical device is positioned in a position of interest, by manipulating the device, a deformable/expandable portion thereof is expanded to engage the walls of the lumen. The deformable portion of the device is formed of curved slits, which allows it to generate a significant force on the walls of the lumen and also allows it to undergo a reverse deformation to the non-expanded state so it can remove freely in the lumen and optionally undergo a further deformation to engage a different portion of the walls of the lumen. The present disclosure also provides an actuation assembly that allows to perform a controllable expansion and reverse deformation of the deformable portion.

### TECHNOLOGICAL FIELD

The present disclosure is in the field of medical devices, in particular medical devices that are intended to expand within a lumen of interest.

### BACKGROUND ART

References considered to be relevant as background to the presently disclosed subject matter are listed below:

WO 2021/234702

Acknowledgement of the above references herein is not to be inferred as meaning that these are in any way relevant to the patentability of the presently disclosed subject matter.

### GENERAL DESCRIPTION

The present disclosure provides a medical device that is capable of performing a controlled expansion of a portion thereof during a medical procedure. The medical device is intended to be inserted into a lumen of interest during the medical procedure, for example a lumen of a blood vessel or a lumen of a lead of a cardiac implemented electronic device (CIED). Once the medical device is positioned in a position of interest, by manipulating the device, a deformable/expandable portion thereof is expanded to engage the walls of the lumen. The deformable portion of the device may be formed of curved slits, which allows it to generate a significant force on the walls of the lumen and also allows it to undergo a reverse deformation to the non-expanded state so it can remove freely in the lumen and optionally undergo a further deformation to engage a different portion of the walls of the lumen. The present disclosure also provides an actuation assembly that allows to perform a controllable expansion and reverse deformation of the deformable portion.

Therefore, an aspect of the present disclosure provides a medical device for use in a medical procedure. The medical device comprising a lumen defined between walls extending between a proximal portion and a distal portion of the device. The device further comprises a deformable portion defined between said proximal portion and said distal portion. It is to be understood that the device is formed of three consecutive portions as part of a single structure—a proximal portion, a deformable portion, and a distal portion. The device further comprises a gripping element that is attached to a portion of the distal portion and extending along said lumen. It is to be noted that the attachment can be directly or indirectly to the walls of the distal portion. The gripping element having a gripping portion for allowing its gripping either by a user using the device or by a device that grips it to perform an action with it. The gripping portion is outside the lumen and is not discernible from other portions of the gripping element. The gripping portion is only defined by its position along the gripping element. The application of force on the deformable portion, by manipulation of the gripping element through the gripping portion, causes it to undergo a deformation that results in a transition from a non-expanded state to a range of expanded states.

It is to be noted that any combination of the described embodiments with respect to any aspect of this present disclosure is applicable. In other words, any aspect of the present disclosure can be defined by any combination of the described embodiments.

In some embodiments of the device, the deformable portion is constituted by or formed with one or more curved slits in said walls. The curved slits are defined as slits that exhibit a certain degree of curvature, which differentiate them from a simple straight, longitudinal slit). The application of force on the deformable portion causes portions of the device between the two adjacent curved slits to expand to a certain degree according to the force extent applied thereon, the expansion degree defines the state in the range of expanded states.

In some embodiments of the device, said curved slits extend along a longitudinal axis of the device. The longitudinal axis is defined between the proximal portion and the distal portion. In some embodiments, the device is tubular, namely the walls defining the lumen creating a tubular shape.

In some embodiments of the device, said curved slits extend along said longitudinal axis in a spiral shape. Namely, the curved slits encircle the lumen as they proceed along the longitudinal axis in a direction towards the distal portion or towards the proximal portion.

In some embodiments of the device, said curved slits encircle the lumen at least one time. In other words, the curved slits complete one or more circles around the lumen.

In some embodiments of the device, the deformable portion is constituted by a plurality of parallel curved slits. In some embodiments of the device, the deformable portion is constituted parallel spiral slits.

In some embodiments of the device, the gripping element is welded to said portion of the distal portion.

In some embodiments of the device, said walls define a tubular shape. Namely, the device is tubular or at least have a tubular portion that includes the deformable portion.

In some embodiments of the device, the gripping element is made of metallic material.

In some embodiments, the device further comprises an actuation assembly configured for controllably causing the deformable portion to undergo said deformation from said non-expanded state to said range of expanded states upon actuation thereof.

In some embodiments of the device, said actuation assembly is anchored to the walls of the proximal portion, either directly or indirectly, and is configured to grip said gripping portion to allow controllable application of pulling force on the gripping element, or controllable pulling of the gripping element to a desired extent, thereby causing controllable deformation of said deformable portion. The pulling is performed towards the proximal direction, namely the pulling of the gripping portion causes the attachment portion of the gripping element to the distal portion to apply force on the deformable portion, thereby causing its deformation.

In some embodiments of the device, said actuation assembly comprises a force adjuster allowing to control the applied pulling force on the gripping element. The force adjuster is adjustable between a maximal force state and a minimal or no-force state. It is to be noted that the force can be increased and decreased, namely after the deformable portion is transitioned to one of the expanded states, by decreasing the force applied on the walls, the deformable portion can transition to a non-expanded state or to an expanded state that is less expanded.

In some embodiments of the device, said actuation assembly comprises a retention unit that is configured to perform said gripping by the actuation assembly, namely to grip the gripping portion and retain it in position.

In some embodiments of the device, said retention unit comprises a first member having a first channel allowing the gripping element to pass therethrough. The first channel comprises a pressable portion that upon application of pressure thereon retain the gripping element in position with respect to the retention unit. The retention unit further comprises a second member having a second channel allowing the gripping element to pass therethrough and is configured to controllably press on said pressable portion to result in said retention of the gripping element, namely the pressing is performed upon actuation of a pressing mechanism of the second member by a user of the device.

In some embodiments of the device, said actuation assembly defines a channel path for passing the gripping element therethrough such that all elements of the actuation assembly surround the gripping element.

In some embodiments, the device further comprises an anchoring element having a tunnel accommodating at least a part of the proximal portion, and optionally a part of the gripping element that extends outside the lumen of the device, and is fixedly attached or welded to walls of the proximal portion. For example, portions of the tunnel are fixedly attached to the walls of the proximal portion. The actuation assembly is anchored to said anchoring element.

Yet another aspect of the present disclosure provides a kit comprising the device of any one of the above-described embodiments or any combination thereof; and an actuation assembly couplable to the gripping portion, wherein said actuation assembly is configured for causing the deformable portion to controllably apply force on the walls towards the distal portion, thereby causing controllable deformation of said deformable portion.

In some embodiments of the kit, said actuation assembly comprises a force adjuster allowing to control the applied pulling force on the gripping element. The force adjuster is adjustable between a maximal force state and a minimal or no-force state. It is to be noted that the force can be increased and decreased, namely after the deformable portion is transitioned to one of the expanded states, by decreasing the force applied on the walls, the deformable portion can transition to a non-expanded state or to an expanded state that is less expanded.

In some embodiments of the kit, said actuation assembly comprises a retention unit that is configured to perform said gripping by the actuation assembly, namely to grip the gripping portion and retain it in position.

In some embodiments of the kit, said retention unit comprises a first member having a first channel allowing the gripping element to pass therethrough. The first channel comprises a pressable portion that upon application of pressure thereon retain the gripping element in position with respect to the retention unit. The retention unit further comprises a second member having a second channel allowing the gripping element to pass therethrough and is configured to controllably press on said pressable portion to result in said retention of the gripping element, namely the pressing is performed upon actuation of a pressing mechanism of the second member by a user of the device.

In some embodiments of the kit, said actuation assembly defines a channel path for passing the gripping element therethrough such that all elements of the actuation assembly surround the gripping element.

In some embodiments of the kit or the device, the device is a lead locking device for insertion into a lumen of a cardiac implemented electronic device (CIED) lead and locking onto or retaining one or more portions of lead walls of the lead lumen.

In some embodiments of the kit or the device, the device is a cardiovascular device for insertion into and expansion of blood vessels.

In some embodiments, the kit or the device are intended for use in a method for performing a medical procedure. The medical procedure comprising: (i) inserting the device into an intended lumen to bring the deformable portion to a first desired position in said intended lumen, the desired position should be understood as any location of interest within the lumen; and (ii) causing a deformation of said deformable portion to result in its expansion in said desired position.

In some embodiments of the kit or the device for use, the intended lumen is a lumen of a cardiac implemented electronic device (CIED) lead.

In some embodiments of the kit or the device for use, said (ii) results in locking of the device in said first desired position, namely the device retains the lead in position, wherein the method further comprising (iii) vibrating the device to thereby resulting transmission of the vibrations to the lead through the first position and causing the lead to vibrate to detach it from tissues and allow its extraction.

In some embodiments of the kit or the device for use, the method further comprising, following said (iii), (iv) collapsing the device into a non-expanded state, in which it is not locked in said first desired position; and (v) moving the device and causing a deformation of said deformable portion to result in its expansion in a second desired position, the second desired position is any location in the lumen that is different than the first position; and (vi) vibrating the device to thereby resulting transmission of the vibrations to the lead through the second position and causing the lead to vibrate to detach it from tissues and allow its extraction.

In some embodiments of the kit or the device for use, the method further comprises (vi) extracting the lead by pulling the device together with the lead it is locked to. The extraction step can either be after a first vibration cycle or after a plurality of vibration cycles in many positions in the lumen of the lead.

In some embodiments of the kit or the device for use, said (iii) or (vi) comprises gripping said gripping portion of the gripping element by a vibration generator and applying the vibrations on it such that the vibrations are transmitted to the lead through the locking.

In some embodiments of the kit or the device for use, said intended lumen is a lumen of a blood vessel.

Yet another aspect of the present disclosure provides an actuation assembly for controllably causing a deformable portion of a medical device to undergo a deformation from said non-expanded state to a range of expanded states upon actuation thereof. The medical device comprises (i) a lumen defined between walls extending between a proximal portion and a distal portion of the device, (ii) a deformable portion defined between said proximal portion and said distal portion, wherein the proximal portion, the deformable portion and the distal portion are consecutive and are a part of a single structure, and (iii) a gripping element attached to a portion of the distal portion and extending along said lumen, the gripping element having a gripping portion that is disposed outside the lumen and is not discernible from other portions of the gripping element, the gripping portion is intended to allow its gripping either by a user using the device or by a device that grips it to perform an action with it. The actuation assembly comprising a coupling member for coupling to the proximal portion and a retention unit that is configured to grip the gripping portion to allow application of pulling force on the gripping element, or controllably pulling of the gripping element to a desired extent by being pushed away from the coupling member, thereby causing deformation of said deformable portion. The pulling is performed towards the proximal direction, namely the pulling of the gripping portion causes the attachment portion of the gripping element to the distal portion to apply force on the deformable portion, thereby causing its deformation. The actuation assembly further comprises a force adjuster allowing to control the applied pulling force on the gripping element. The force adjuster is adjustable between a maximal force state and a minimal or no-force state. It is to be noted that the force can be increased and decreased, namely after the deformable portion is transitioned to one of the expanded states, by decreasing the force applied on the walls, the deformable portion can transition to a non-expanded state or to an expanded state that is less expanded. The force adjuster controls the extent to which the retention unit is pushed away from the coupling member, the further it is pushed away, the greater the pulling force is and therefore the greater the deformation. The greater the deformation, the greater the anchoring force of the medical device to the walls of the lumen.

In other words, the coupling member is coupled to the proximal portion while the retention unit grips the gripping portion. The coupling member and the retention unit are being a part of the same assembly, therefore by changing the distance between the coupling member and the retention unit, a pulling force is applied on the gripping element that results in the deformation of the deformable portion. The change of the distance between the coupling member and the retention unit is performed and controlled by the force adjuster.

In some embodiments of the actuation assembly, the retention unit comprises a first member having a first channel allowing the gripping element to pass therethrough, wherein said first channel comprises a pressable portion that upon application of pressure thereon retains the gripping element in position with respect to the retention unit. The retention unit further comprises a second member having a second channel allowing the gripping element to pass therethrough and is configured to controllably press on said pressable portion to result in said retention of the gripping element.

In some embodiments of the actuation assembly, the actuation assembly defines a channel path for passing the gripping element therethrough such that all elements of the actuation assembly surround the gripping element.

In some embodiments of the actuation assembly, the coupling member comprises a coupling member channel for allowing a portion of the proximal portion, or a portion of the gripping element, or both to pass therethrough, therefore allowing the gripping portion to reach the retention unit.

In some embodiments of the actuation assembly, the force adjuster comprises a first adjusting member for allowing setting a selected distance of the retention unit from the coupling member and a second adjusting member for securing the first adjusting member in said selected distance.

In some embodiments of the actuation assembly, the first adjusting member comprises a force indicator that indicates the extent of the distance between the coupling member and the retention unit. This can be done by a view port of the first adjusting member that shows the position of the first adjusting member on a screw it is screwed on. The setting of the force by the first adjusting member is performed by changing its position on the screw.

In some embodiments, the actuation assembly further comprises a common thread member for screwing the coupling member and the force adjuster thereto, the common thread member comprises a common thread channel for allowing a portion of the proximal portion, or a portion of the gripping element, or both to pass therethrough, wherein a position of the force adjuster on said common thread member defines the extent of the pulling force.

In some embodiments of the actuation assembly, the common thread member is part of the coupling member, namely integral therewith. The common thread member is extending towards the direction of the retention unit.

Yet another aspect of the present disclosure provides a lead locking device for insertion into a lumen of a cardiac implemented electronic device (CIED) lead and locking onto or retaining one or more portions of lead walls of the lead lumen. The lead locking device comprising: (i) a lumen defined between walls extending between a proximal portion and a distal portion of the lead locking device; (ii) a deformable portion defined between said proximal portion and said distal portion; and (iii) a gripping element attached to a portion of the distal portion and extending along said lumen, the gripping element having a gripping portion for gripping it. The deformable portion is constituted by one or more curved slits in said walls such that application of force on the deformable portion causes it to undergo a deformation from a non-expanded or non-lead-engaging state to a range of expanded, lead-engaging states. The application of force on the deformable portion causes portions of the lead locking device between two adjacent curved slits to expand and engage the walls of the lumen of the lead, thereby locking to it in position.

### EMBODIMENTS

The following are optional embodiments and combinations thereof in accordance with aspects of the present disclosure:
1. A medical device, comprising:
   a lumen defined between walls extending between a proximal portion and a distal portion of the device;
   a deformable portion defined between said proximal portion and said distal portion;
   a gripping element attached to a portion of the distal portion and extending along said lumen, the gripping element having a gripping portion for gripping it;
   wherein application of force on the deformable portion, by manipulation of the gripping element through the gripping portion, causes it to undergo a deformation that results in a transition from a non-expanded state to a range of expanded states.
2. The device of embodiment 1, wherein the deformable portion is constituted by one or more curved slits in said walls; and
3. The device of embodiment 2, wherein said curved slits extend along a longitudinal axis of the device.
4. The device of embodiment 3, wherein said curved slits extend along said longitudinal axis in a spiral shape.
5. The device of embodiment 4, wherein said curved slits encircle the lumen at least one time.
6. The device of any one of embodiments 1-5, wherein the deformable portion is constituted by a plurality of parallel curved slits.
7. The device of any one of embodiments 1-6, wherein the gripping element is welded to said portion of the distal portion.
8. The device of any one of embodiments 1-7, wherein said walls define a tubular shape.
9. The device of any one of embodiments 1-8, wherein the gripping element is made of metallic material.
10. The device of any one of embodiments 1-9, comprising an actuation assembly configured for controllably causing the deformable portion to undergo said deformation from said non-expanded state to said range of expanded states upon actuation thereof.
11. The device of embodiment 9, wherein said actuation assembly is anchored to the proximal portion and is configured to grip said gripping portion to allow controllable application of pulling force on the gripping element, thereby causing controllable deformation of said deformable portion.
12. The device of embodiment 10 or 11, wherein said actuation assembly comprises a force adjuster allowing to control the applied pulling force on the gripping element.
13. The device of any one of embodiments 10-12, wherein said actuation assembly comprises a retention unit that is configured to perform said gripping by the actuation assembly.
14. The device of embodiment 13, wherein said retention unit comprises
   a first member having a first channel allowing the gripping element to pass therethrough, wherein said first channel comprises a pressable portion that upon application of pressure thereon retain the gripping element in position with respect to the retention unit,
   a second member having a second channel allowing the gripping element to pass therethrough and is configured to controllably press on said pressable portion to result in said retention of the gripping element.
15. The device of any one of embodiments 10-14, wherein said actuation assembly defines a channel path for passing the gripping element therethrough.
16. The device of any one of embodiments 10-15, comprising an anchoring element having a tunnel accommodating at least a part of the proximal portion and is fixedly attached to the proximal portion, wherein said actuation assembly is anchorable to said anchoring element.
17. A kit comprising:
   the device of any one of embodiments 1-9; and
   an actuation assembly couplable to the gripping portion, wherein said actuation assembly is configured for causing the deformable portion to controllably apply force on the walls towards the distal portion, thereby causing controllable deformation of said deformable portion.
18. The kit of embodiment 17, wherein said actuation assembly is any one of that defined in embodiments 10-16.
19.The device or kit of any one of embodiments 1-18, wherein the device is a lead locking device for insertion into a lumen of a cardiac implemented electronic device (CIED) lead and locking onto one or more portions of lead walls of the lead lumen.
20. The device or kit of any one of embodiments 1-19, wherein the device is a cardiovascular device for insertion into and expansion of blood vessels.
21. The device or kit of any one of embodiments 1-20 for use in a method for performing a medical procedure, comprising:
   inserting the device into an intended lumen to bring the deformable portion to a first desired position in said intended lumen; and
   causing a deformation of said deformable portion to result in its expansion in said desired position.
22. The device or kit for use of embodiment 21, wherein said intended lumen is a lumen of a cardiac implemented electronic device (CIED) lead.
23.The device or kit for use of embodiment 22, wherein said causing results in locking of the device in said first desired position, wherein the method further comprising vibrating the device to thereby resulting transmission of the vibrations to the lead through the first position.
24. The device or kit for use of embodiment 23, wherein the method further comprising, following said vibrating, collapsing the device into a non-expanded state, in which it is not locked in said first desired position; and
   moving the device and causing a deformation of said deformable portion to result in its expansion in a second desired position; and
   vibrating the device to thereby resulting transmission of the vibrations to the lead through the second position.
25. The device or kit for use of embodiment 23 or 24, wherein the method further comprising extracting the lead by pulling the device.
26. The device or kit for use of any one of embodiments 23-25, wherein said vibrating comprises gripping said gripping portion of the gripping element by a vibration generator and applying the vibrations on it.
27. The device or kit for use of embodiment 21, wherein said intended lumen is a lumen of a blood vessel.
28. An actuation assembly for controllably causing a deformable portion of a medical device to undergo a deformation from said non-expanded state to a range of expanded states upon actuation thereof, said medical device comprises
   (i) a lumen defined between walls extending between a proximal portion and a distal portion of the device, (ii) a deformable portion defined between said proximal portion and said distal portion, and (iii) a gripping element attached to a portion of the distal portion and extending along said lumen, the gripping element having a gripping portion for gripping it, said actuation assembly comprising:
   a coupling member for coupling to the proximal portion;
   a retention unit that is configured to grip the gripping portion to allow application of pulling force on the gripping element, thereby causing deformation of said deformable portion;
   a force adjuster allowing to control the applied pulling force on the gripping element.
29. The actuation assembly of embodiment 28, wherein the retention unit comprises
   a first member having a first channel allowing the gripping element to pass therethrough, wherein said first channel comprises a pressable portion that upon application of pressure thereon retains the gripping element in position with respect to the retention unit,
   a second member having a second channel allowing the gripping element to pass therethrough and is configured to controllably press on said pressable portion to result in said retention of the gripping element.
30. The actuation assembly of embodiment 28 or 29, wherein said actuation assembly defines a channel path for passing the gripping element therethrough.
31. The actuation assembly of any one of claims 28-30, wherein the coupling member comprises a coupling member channel for allowing a portion of the proximal portion, or a portion of the gripping element, or both to pass therethrough.
32. The actuation assembly of any one of claims 28-31, wherein the force adjuster comprises a first adjusting member for allowing setting a selected distance of the retention unit from the coupling member and a second adjusting member for securing the first adjusting member in said selected distance.
33. The actuation assembly of claim 32, wherein the first adjusting member comprises a force indicator that indicates the extent of the distance between the coupling member and the retention unit.
34. The actuation assembly of any one of claims 28-33, comprising a common thread member for screwing the coupling member and the force adjuster thereto, the common thread member comprises a common thread channel for allowing a portion of the proximal portion, or a portion of the gripping element, or both to pass therethrough, wherein a position of the force adjuster on said common thread member defines the extent of the pulling force.
35. The actuation assembly of claim 34, wherein the common thread member is part of the coupling member extending towards the retention unit.
36. A lead locking device for insertion into a lumen of a cardiac implemented electronic device (CIED) lead and locking onto one or more portions of lead walls of the lead lumen, comprising:
   a lumen defined between walls extending between a proximal portion and a distal portion of the lead locking device;
   a deformable portion defined between said proximal portion and said distal portion;
   a gripping element attached to a portion of the distal portion and extending along said lumen, the gripping element having a gripping portion for gripping it;
   wherein the deformable portion is constituted by one or more curved slits in said walls such that application of force on the deformable portion causes it to undergo a deformation from a non-expanded state to a range of expanded, lead-engaging states.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are schematic illustrations of different views of a non-limiting example of an embodiment of the medical device for use in a medical procedure according to an aspect of the present disclosure. FIG. 1A is a side view; FIG. 1B is a perspective view; and FIG. 1C is a cross-sectional view.

FIG. 2A-2C are schematic illustrations of different examples of embodiments of the medical device, each presenting different characteristics of the curved slits.

FIG. 3A-3E are schematic illustrations of different states of a non-limiting example of an actuation assembly according to an aspect of the present disclosure.

FIG. 4 is a schematic illustration of a non-limiting example of an anchoring element welded to the proximal portion of the medical device of the present disclosure.

### DETAILED DESCRIPTION

The following figures are provided to exemplify embodiments and realization of the invention of the present disclosure.

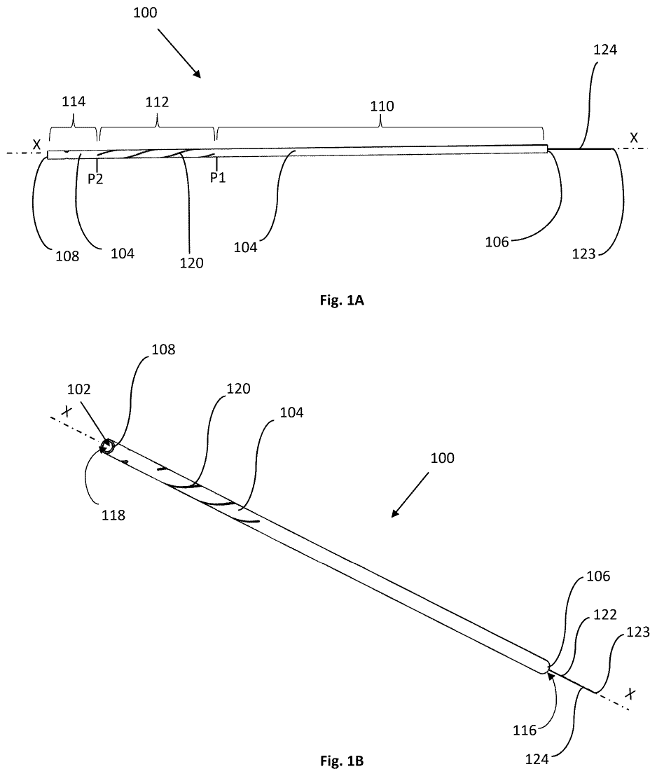

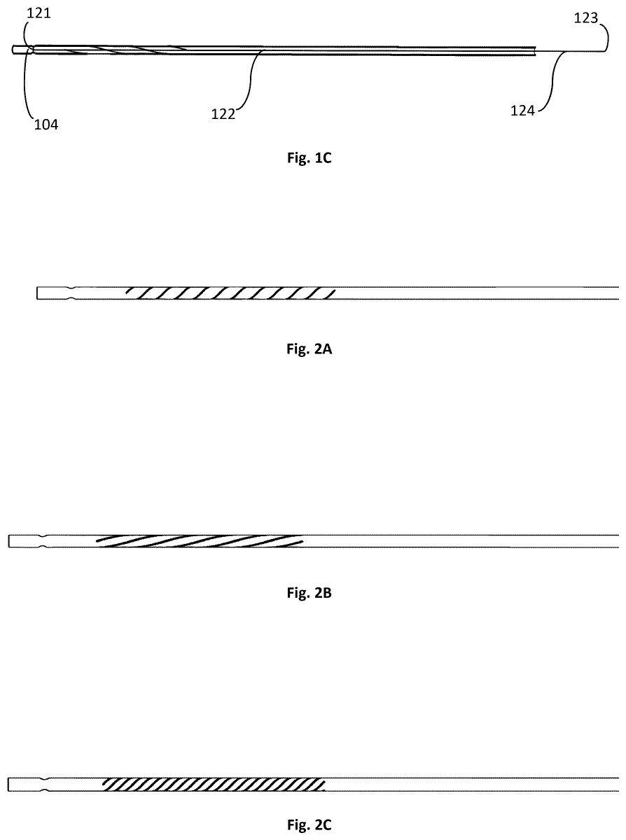

Reference is first being made to FIGS. 1A-1C, which are schematic illustrations of different views of a non-limiting example of an embodiment of the medical device for use in a medical procedure according to an aspect of the present disclosure. The device 100 comprises a lumen 102 defined by walls 104 creating an elongated tubular structure. The device 100 is extending between a proximal end 106 and a distal end 108 and defining three consecutive portions-a proximal portion 110, a deformable portion 112 and a distal portion 114. A proximal opening 116 and a distal opening 118 are defined in the proximal end 106 and the distal end 108, respectively, such that the lumen 102 is accessible from both sides of the device 100. It is to be noted, that in some embodiments, the distal opening 118 does not exist and the distal end 108 is sealed. The deformable portion 112 is sandwiched between the proximal portion 110 and the distal portion 114 and is formed with a plurality of curved slits 120, each extending between a first longitudinal position P1 and a second longitudinal position P2, the longitudinal positions are defined along a longitudinal axis X defined the lumen 102 of the device 100. It is to be noted that each slit may start and end at a different longitudinal position than other slits, though in this example, the start and end longitudinal position of each slit is similar, though the start and end angular positions are different and the slits are parallel to one another. Each curved slit is spiraling on the walls 104 so it encircles at least a portion of the lumen 102 or completes one or more encircling cycles of the lumen 102. This slits configuration results that a force that applied on the deformable portion 112 causing it to deform such that the portions between two slits extending outwardly and expanding the volumetric size of the deformable portion 112 and effectively transitioning the device from a non-expanded state to one of the expanded states from a range of expanded states. In the expanded state, the device can fulfill one of its purposes, whether it is an expansion of a blood vessel or locking in position in a lumen of a lead of a cardiac implemented electronic device (CIED), such as a pacemaker. The amount of applied force on the deformable portion 112 defines the expanded state the device is found in. The range of expanded states may differ one from the other by the amount of pressure that the deformable portion 112 applies on its surrounding, whether it is walls of the lead or walls of a blood vessel.

The density of the slits, namely the number of parallel slits, and their curvature profile, namely a characterization that defines how many circles the slits make in a defined progress along the longitudinal axis, can be varied and may be selected according to the desired characteristics of the medical device. FIGS. 2A-2C show different optional and non-limiting examples of embodiments of the medical device, each presenting different characteristics of the curved slits.

A gripping element 122 is extending between a first end 121, that is attached to walls 104 of the distal portion 114, either directly or indirectly, and a second end 123. The gripping element 122 is extending through the lumen 102 until it protrudes through the proximal opening 116 such that the second end 124 is positioned outside of the lumen 102, as can be best seen in FIG. 1C. A gripping portion 124 is defined as the exposed portion of the gripping element 122 that is outside of the lumen 102. It is to be noted that the gripping portion 124 is not necessarily discernible from other portions of the gripping element 122. By gripping the gripping portion 124 and pulling it towards the proximal direction PD, a force is applied on the deformable portion 112 and causes it to deform from a non-expanded state to one of its expanded states. The gripping element 122 is in the form of a metallic thread and it can either be manipulated by a user or by an actuation assembly that is configured to pull it in a controllable manner so as to allow to bring the device 100 to a desired expanded state.

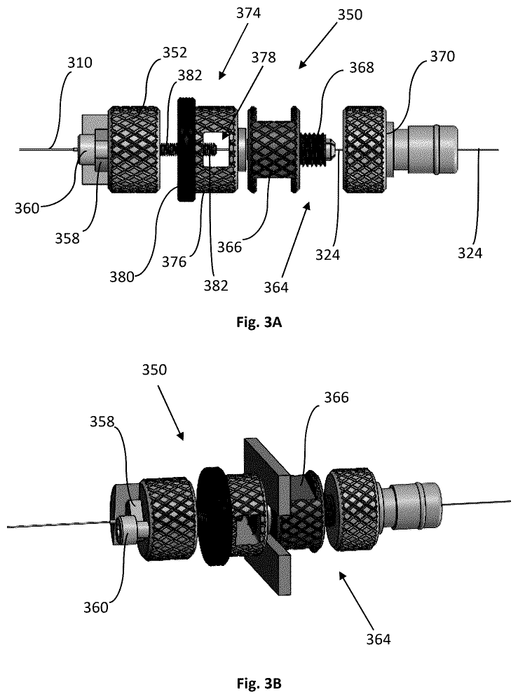

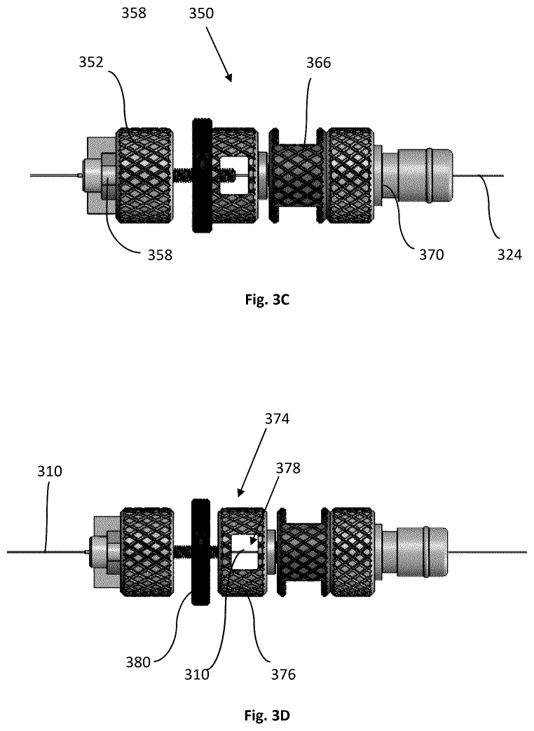

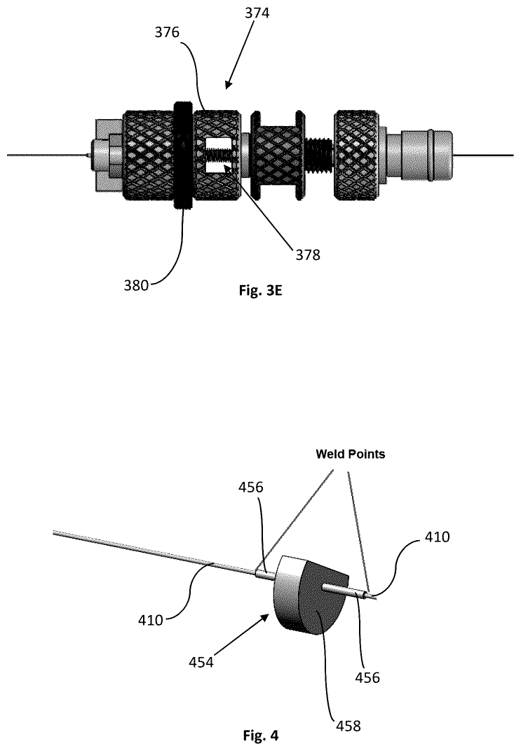

In the figures throughout the application, like elements of different figures were given similar reference numerals shifted by the number of hundreds corresponding to the number of the respective figure. For example, element 310 in FIGS. 3A-3E serves the same function as element 110 in FIGS. 1A-1C.

FIGS. 3A-3E are schematic illustrations of a non-limiting example of an actuation assembly according to an aspect of the present disclosure. It is to be noted that the actuation assembly is not limited to be used with the medical device that is described above, as it can be coupled to any other medical device with a different type of deformable portion. As can be appreciated, the actuation assembly 350 is fitted around the gripping portion, therefore it defines a channel path through all of its elements that the gripping portion is inserted through. The actuation assembly 350 comprises a coupling member 352 that is configured to allow coupling of part of the actuation assembly 350 to the proximal portion 310 of the medical device.

Since that gripping element can be very small (less than 1 millimeter), in some embodiments of the medical device, an anchoring element 454 is fixedly attached or welded to the proximal portion 410, as can be seen in FIG. 4, for facilitating the coupling of the coupling member 352. The anchoring element 454 defines a tunnel 456 in which a part of the proximal portion 410 resides, and at times also a part of the gripping portion. An anchoring member 458 is formed around the tunnel 456 to allow the coupling member 352 of the actuation assembly to be coupled and anchored thereto, therefore, allow it to indirectly be coupled to the proximal portion 410. In this example, the anchoring member 458 is in the form of a trimmed disc for reasons explained below.

Reference is made again to FIGS. 3A-3E. The coupling member 352 is placed next to the anchoring member 358 and a screw element 360 is screwed to a screwing thread formed in the coupling member 352 that remains exposed due to the trimmed shape of the anchoring member 358, as can be best seen in FIG. 3B. The screwing element 360 is screwed until its head 362 firmly engages with the anchoring member 358, thereby effectively anchoring the coupling member 352 to the proximal portion 310. It is to be noted that the anchoring member 358 may have any shape that leave the screwing thread exposed and allows the head of the screwing element to engage with it.

The actuation assembly 350 further comprises a retention unit 364 configured for gripping the gripping portion 324 to allow application of pulling force on the gripping element. The retention unit 364 comprises a first member 366 having a first channel allowing the gripping element to pass therethrough. The first channel comprises pressable portion 368, that upon application of pressure thereon, retains the gripping element in position with respect to the retention unit 364. A second member 370 of the retention unit 364 has a second channel allowing the gripping element to pass therethrough and is configured to controllably press on said pressable portion to result in said retention of the gripping element. The controllably press is performed either by fitting the second member 370 over the pressable portion 368 or by screwing it on a pressing thread 372 that is formed around the pressable portion 368, as can be seen in FIG. 3C.

The actuation assembly 350 further comprise a force adjuster 374 that allows to control the applied pulling force on the gripping element. The force adjuster is disposed between the retention unit 364 and the coupling member 352. Once the coupling member 352 is (i) coupled and anchored to the proximal portion 310 of the device or to an anchoring element that is fixed to the proximal portion and (ii) the retention unit 364 retains the gripping portion 324, the force adjuster 374 can be adjusted to define the applied pulling force on the gripping element and therefore the extent of the deformation of the deformable portion of the medical device. The force adjuster 374 controls the extent that the retention unit 364 is pushed away from the coupling member 352, the further it is pushed away, the greater the pulling force and therefore the deformation. The force adjuster 374 comprises a first adjusting member 376 that initially sets the force application state, and it comprises an aperture 378 through which the extent of the force application can be inferred. A second adjusting member 380 of the force adjuster 374 is configured to secure the position of the first adjusting member 376 in place. For example, FIG. 3D shows the first adjusting member 376 of the force adjuster 374 in a position that defines a maximal force application, though the second adjusting member 380 is not in a securing position, namely adjacent to the first adjusting member 376; and FIG. 3E shows the first adjusting member 376 of the force adjuster 374 in a state that applies minimum force, and the second adjusting member 380 is securing the state by being adjacent to the first adjusting member 376. It is to be noted that the coupling member 352 and the force adjuster 374 are threaded on a common thread member 382. Therefore, the adjustment of the force adjuster 374 is performed by positioning it on different positions along the thread member 382. The thread member 382 also comprises a channel that defines a part of the channel path. The first member 366 is fixedly coupled to the first adjusting member 376, therefore forming a coupled structure of the coupling member, the force adjuster, and a part of the retention unit. In some embodiments, the thread member 382 can be part of the coupling member 352.

1-27. (canceled)

28. An actuation assembly for controllably causing a deformable portion of a medical device to undergo a deformation from a non-expanded state to a range of expanded states upon actuation thereof, said medical device comprises:
(i) a lumen defined between walls extending between a proximal portion and a distal portion of the device, (ii) a deformable portion defined between said proximal portion and said distal portion, and (iii) a gripping element attached to a portion of the distal portion and extending along said lumen, the gripping element having a gripping portion for gripping it, said actuation assembly comprising:
a coupling member for coupling to the proximal portion;
a retention unit that is configured to grip the gripping portion to allow application of pulling force on the gripping element by being pushed away from the coupling member, thereby causing deformation of said deformable portion;
a force adjuster allowing to control the applied pulling force on the gripping element.

29. The actuation assembly of claim 28, wherein the retention unit comprises
a first member having a first channel allowing the gripping element to pass therethrough, wherein said first channel comprises a pressable portion that upon application of pressure thereon retains the gripping element in position with respect to the retention unit,
a second member having a second channel allowing the gripping element to pass therethrough and is configured to controllably press on said pressable portion to result in said retention of the gripping element.

30. The actuation assembly of claim 28, wherein said actuation assembly defines a channel path for passing the gripping element therethrough.

31. The actuation assembly of claim 28, wherein the coupling member comprises a coupling member channel for allowing a portion of the proximal portion, or a portion of the gripping element, or both to pass therethrough.

32. The actuation assembly of claim 28, wherein the force adjuster comprises a first adjusting member for allowing setting a selected distance of the retention unit from the coupling member and a second adjusting member for securing the first adjusting member in said selected distance.

33. The actuation assembly of claim 32, wherein the first adjusting member comprises a force indicator that indicates the extent of the distance between the coupling member and the retention unit.

34. The actuation assembly of claim 28, comprising a common thread member for screwing the coupling member and the force adjuster thereto, the common thread member comprises a common thread channel for allowing a portion of the proximal portion, or a portion of the gripping element, or both to pass therethrough, wherein a position of the force adjuster on said common thread member defines the extent of the pulling force.

35. The actuation assembly of claim 34, wherein the common thread member is part of the coupling member extending towards the retention unit.

36. A kit comprising:
A medical device that comprises:
a lumen defined between walls extending between a proximal portion and a distal portion of the device;
a deformable portion defined between said proximal portion and said distal portion;
a gripping element attached to a portion of the distal portion and extending along said lumen, the gripping element having a gripping portion for gripping it;
wherein application of force on the deformable portion, by manipulation of the gripping element through the gripping portion, causes it to undergo a deformation that results in a transition from a non-expanded state to a range of expanded states.; and
an actuation assembly according to claim 28.

37. The kit of claim 36, wherein the deformable portion is constituted by one or more curved slits in said walls.

38. The kit of claim 36, wherein said curved slits extend along a longitudinal axis of the device; wherein said curved slits extend along said longitudinal axis in a spiral shape; wherein said curved slits encircle the lumen at least one time.

39. The kit of claim 36, wherein the deformable portion is constituted by a plurality of parallel curved slits.

40. The kit of claim 36, wherein the gripping element is welded to said portion of the distal portion; wherein said walls define a tubular shape; and wherein the gripping element is made of metallic material.

41. The kit of claim 36, wherein the device is a lead locking device for insertion into a lumen of a cardiac implemented electronic device (CIED) lead and locking onto one or more portions of lead walls of the lead lumen; and wherein the device is a cardiovascular device for insertion into and expansion of blood vessels.

42. A method for performing a medical procedure, comprising:
providing the kit of claim 36;
inserting the device into an intended lumen to bring the deformable portion to a first desired position in said intended lumen; and
causing a deformation of said deformable portion to result in its expansion in said desired position.

43. The method of claim 42, wherein said intended lumen is a lumen of a cardiac implemented electronic device (CIED) lead; and wherein said intended lumen is a lumen of a blood vessel.

44. The method of claim 42, wherein said causing results in locking of the device in said first desired position, wherein the method further comprising vibrating the device to thereby resulting transmission of the vibrations to the lead through the first position; wherein the method further comprising, following said vibrating, collapsing the device into a non-expanded state, in which it is not locked in said first desired position; and
moving the device and causing a deformation of said deformable portion to result in its expansion in a second desired position; and
vibrating the device to thereby resulting transmission of the vibrations to the lead through the second position.

45. The method of claim 42, further comprising extracting the lead by pulling the device.

46. The method of claim 42, wherein said vibrating comprises gripping said gripping portion of the gripping element by a vibration generator and applying the vibrations on it.

47. The method of claim 42, wherein the kit further comprises an anchoring element having a tunnel accommodating at least a part of the proximal portion and is fixedly attached to the proximal portion, wherein said actuation assembly is anchorable to said anchoring element.
